(19) United States
(12) Patent Application Publication
(10) Pub. No.: US 2022/0198419 A1
(43) Pub. Date: Jun. 23, 2022

(54) SYSTEM AND METHOD FOR MANAGING PAYMENTS FOR ACCESSING PATIENTS' INFORMATION

(71) Applicant: Patientory, Inc., Atlanta, GA (US)

(72) Inventor: Chrissa Tanelia McFarlane, Atlanta, GA (US)

(21) Appl. No.: 17/607,207

(22) PCT Filed: Jun. 10, 2019
(86) PCT No.: PCT/US2019/036416

§ 371 Date: Oct. 28, 2021

Related U.S. Application Data
(60) Provisional application No. 62/683,513, filed on Jun. 11, 2018.
(60) Provisional application No. 62/683,524, filed on Jun. 11, 2018.
(60) Provisional application No. 62/683,537, filed on Jun. 11, 2018.
(60) Provisional application No. 62/683,556, filed on Jun. 11, 2018.
(60) Provisional application No. 62/683,568, filed on Jun. 11, 2018.

(51) Int. Cl. G06Q 20/14; G06Q 20/40; G16H 10/60; G06F 21/62

(52) U.S. Cl. CPC G06Q 20/14; G06Q 20/40; G06Q 2220/00; G06F 21/6245; G16H 10/60

(57) ABSTRACT

A system and a method for managing payments for accessing patients' information are disclosed. The method includes receiving a request from a third party user. The request corresponds to accessing the patients information stored in a blockchain database. The method further includes determining at least one type of the patients' information requested by the third party user. Further, the method includes receiving an authorization from the patient to provide an access of the patients information to the third party user. Thereafter, a payment is calculated based at least on the at least one type of the patients information and the authorization. Finally, access may be provided to the patients information upon receiving the payment.

### CROSS REFERENCE TO RELATED APPLICATIONS

The present disclosure is related and claims priority under 35 U.S.C. § 1.119(e) to U.S. provisional application Nos. 62/683,513, entitled SYSTEM AND METHOD FOR MANAGING PAYMENTS FOR ACCESSING PATIENTS INFORMATION; 62/683,524, entitled SYSTEM AND METHOD OF CONTROLLING ACCESS OF A USERS HEALTH INFORMATION STORED OVER A HEALTH CARE NETWORK; 62/683,537, entitled SYSTEM AND METHOD FOR REGULATING A VALUE OF A CRYPTOCURRENCY USED IN A HEALTH CARE NETWORK, 62/683,556, entitled SYSTEM AND METHOD FOR FACILITATING PAYMENT REQUESTS WITHIN A HEALTH CARE NETWORK, and 62/683,568, entitled SYSTEM AND METHOD OF MANAGING ACCESS OF A USER'S HEALTH INFORMATION STORED OVER A HEALTH CARE NETWORK, all filed on Jun. 11, 2018, to Chrissa Tanelia McFarlane, the contents of all of which are hereby incorporated by reference in their entirety, for all purposes.

### FIELD OF THE DISCLOSURE

The present disclosure is generally related to a payment management system, and more particularly related to managing payments for accessing patients' information, over a blockchain network.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner The blocks are added to the blockchain in a linear and chronological order. Various types of information, such as patient information, can be stored in a blockchain database.

Currently, patient information is easily accessible to various entities such as hospitals, insurers, or contact research organizations. Such access to the information may lead to its misuse by the different entities. Therefore, there is a need for a method for controlling access to the patient information and a method of managing payments made for accessing the patient information.

### SUMMARY

In a first embodiment, a computer-implemented method for managing payments for accessing patients' information includes receiving, from a third party user, a request to access a patient's information stored in a blockchain database and determining a type of the patients' information. The computer-implemented method also includes receiving an authorization from a patient to provide an access of the patients' information to the third party user, calculating, based at least on the type of the patients' information and the authorization from the patient, a payment value to be paid by the third party user for the patient's information, and allowing the access to the patients' information to the third party user upon receipt of the payment value.

In a second embodiment, a system for managing payments for accessing patients' information includes a memory storing instructions and one or more processors configured to execute the instructions. The instructions cause the system to receive, from a third party user, a request to access a patient's information stored in a blockchain database, to determine a type of the patients' information, and to receive an authorization from a patient to provide an access of the patients' information to the third party user. The instructions also cause the system to calculate, based at least on the type of the patients' information and the authorization from the patient, a payment value to be paid by the third party user for the patient's information, and to allow the access to the patients' information to the third party user upon receipt of the payment value.

In yet other embodiments, a computer-implemented method for accessing a patient information in a health information exchange includes requesting, via a communication network, the patient information from the health information exchange. The computer-implemented method also includes receiving, from a server in the health information exchange, a condition for access when an access to the patient information is approved, authorizing a transaction to access the patient information when the condition is fulfilled, and receiving an address in a blockchain database, and a key to access a document in the address, wherein the document includes at least a portion of the patient information as determined by the condition for access.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram 100 of a system 102, according to various embodiments.

FIG. 2A illustrates a method for symmetric encryption of data, according to various embodiments.

FIG. 2B illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in a health care network implemented, for example, over a blockchain network, according to various embodiments.

FIG. 6A illustrates an example of a table showing various example types of information stored in a subscriber database, according to various embodiments.

FIG. 6B illustrates an example of a table showing various example types of information stored in a smart contracts database, according to various embodiments.

FIG. 6C illustrates an example of a table showing various example types of information stored in an access rights database, according to various embodiments.

FIG. 6D illustrates an example of a table showing various example types of information stored in a patient database, according to various embodiments.

FIG. 7 illustrates a flowchart showing an example method that can be carried out by a third party interface of a third party device, according to various embodiments.

FIG. 8 illustrates a flowchart 800 showing an example method that can be carried out by a patient interface present on a user device, according to various embodiments.

FIG. 9 illustrates a flowchart showing a method that can be performed by a setup module, according to various embodiments.

FIG. 10 illustrates a flowchart showing a method that can be performed by an access module, according to various embodiments.

FIG. 11 illustrates a flowchart showing a method that can be performed by a payment module, according to various embodiments.

FIG. 12 illustrates a flowchart showing a method that can be performed by a patient authorization module, according to various embodiments.

FIG. 13 is a block diagram that illustrates a computer system used to perform at least some of the steps and methods in accordance with various embodiments.

### DETAILED DESCRIPTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, particular embodiments of the systems and methods will be described.

Current systems and methods for storing and managing transfer of health information between multiple parties in the healthcare system are often centralized structures subject to hacking, and yet mired in strict security regulations and onerous overhead costs. This state of affairs leads to a lack of efficient and transparent information exchange, to the ultimate detriment of patients and physicians Embodiments as disclosed herein resolve the above technical problem arising in the realm of healthcare data management by implementing a blockchain infrastructure to minimize security breaches and facilitate coordination between multiple entities and organizations, thus improving the health outcomes for patients.

In some embodiments, a blockchain infrastructure as disclosed herein allows the care providers to avoid medication errors, thus reducing the need for duplicate testing. Further, blockchain technology as disclosed herein effectively tracks and timestamps activities related to health information data. Thus, some embodiments provide a robust audit trail that ensures access to all interested and authorized parties to an updated version of a medical record.

Furthermore, in some embodiments, a blockchain network as disclosed herein includes smart contracts configured with universal parameters. Accordingly, patients become the primary intermediaries for sending and receiving health information. Records stored in a blockchain network as disclosed herein are robust to tampering or error, and are stored across multiple participating users (e.g., the entire blockchain network). Accordingly, recovery contingencies are unnecessary. Moreover, the transparency of a blockchain network as disclosed herein substantially reduces the number of data exchange integration points and the need for tedious reporting activities.

In some embodiments, a mobile application installed in client devices allow users to interact with the blockchain network and access features such as messaging, and access updated and accurate health information. Further, some embodiments provide tracking applications and other activity trackers to enable doctors, care providers, and other parties in the blockchain network to communicate on a single, easy to use platform. Furthermore, in some embodiments, artificial intelligence, machine learning, neural networks, and other nonlinear algorithms are incorporated to store and manage data in the blockchain network.

Some embodiments provide the ability for patients and other users of the blockchain network to access tokens from an external blockchain to convert into a supported cryptocurrency for access and use of storage features.

Embodiments of the present disclosure will be described more fully hereinafter with reference to the accompanying drawings in which like numerals may represent like elements throughout the several figures, and in which various example embodiments are shown. Embodiments may, however, be embodied in many different forms and should not be construed as limited to the embodiments set forth herein. The examples set forth herein are non-limiting examples and are merely examples among other possible examples.

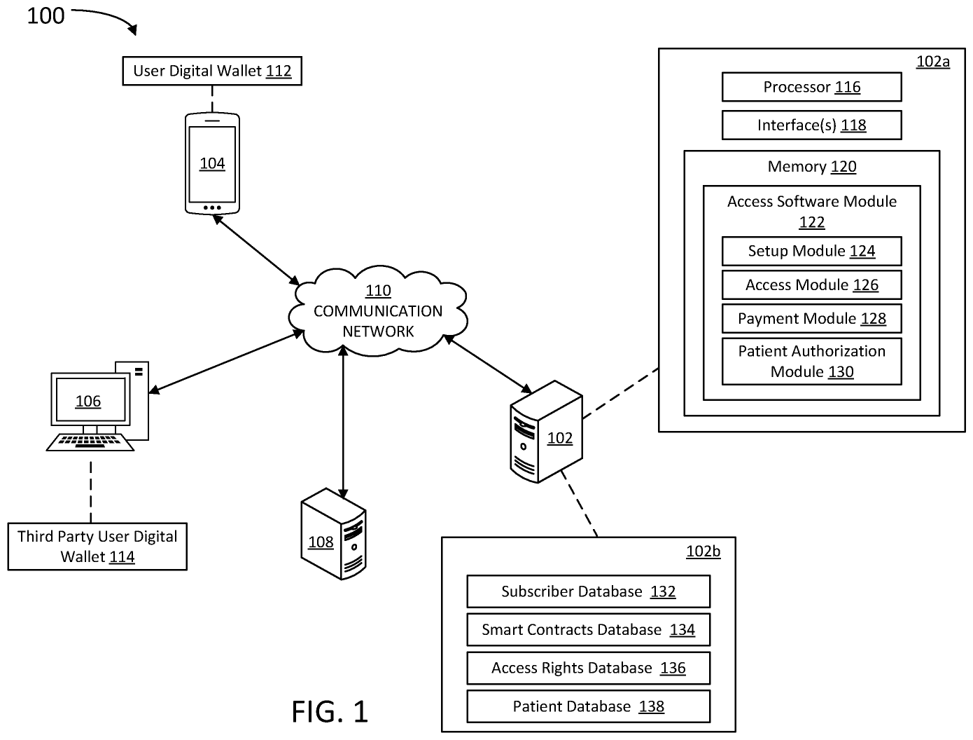

FIG. 1 illustrates a network connection diagram 100 of a Health Information Exchange (HIE) system 102 for managing payments for accessing information including, for example, patient information (hereinafter referred to as ‘patient information’). The HIE system 102 may include one or more user interfaces. The one or more user interfaces may be accessed by one or more users via one or more user devices 104. The HIE system 102 may be connected with a user device 104, a third party device 106, and a financial platform (e.g., coin market) 108, through a communication network 110.

The user device 104 may be operated by one or more patients for allowing an access to patient information. The user device 104 may have a user digital wallet 112. Although user device 104 is shown as a smart phone for illustrative purposes only, user device 104 can be, for example, a laptop, desktop, tablet, and a phablet.

In accordance with various embodiments, the third party device 106 may be used, for example, by a third party user or a subscriber for requesting the patient information. The third party device 106 may be used to access the patient information using a third party interface. Further, the third party user may be required to pay for accessing the patient information using a third party user digital wallet 114. It should be noted that the third party device 106, operated by the third party user, may be operated by a party belonging to, for example, a hospital, an insurance company, a pharmaceutical company, or a Contract Research Organization (CRO). Although the third party device 106 is shown as a desktop for illustrative purposes only, third party device 106 could be, for example, a smart phone, tablet, and a phablet.

In various embodiments, the financial platform (e.g., coin market) 108 may verify and facilitate changes in balance of subscriber's and patient's accounts as well as facilitate payments to a patient network host.

The communication network 110 may be a wired and/or a wireless network. The communication network 110, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may include a group of components 102a for managing payments for accessing patients' information. The group of components 102a may include a processor 116, interface(s) 118, and a memory 120. In various embodiments, the memory 120 may include an access software module 122. The access software module 122 may include a setup module 124, an access module 126, a payment module 128, and a patient authorization module 130.

The processor 116 may execute an algorithm stored in the memory 120 for managing payments for accessing patient information. The processor 116 may also be configured to decode and execute any instructions received from one or more other electronic devices or server(s). The processor 116 may include one or more general purpose processors (e.g., microprocessors) and/or one or more special purpose processors (e.g., digital signal processors (DSPs) or System On Chips (SOCs), Field Programmable Gate Arrays (FPGAs), or Application-Specific Integrated Circuits (ASICs)). The processor 116 may be configured to execute one or more computer-readable program instructions, such as program instructions to carry out any of the functions described in this description.

The interface(s) 118 may help an operator to interact with the HIE system 102. The interface(s) 118 may either accept inputs from users or provide outputs to the users, or may perform both the actions. In one case, a user may interact with the interface(s) 118 using one or more user-interactive objects and devices. The user-interactive objects and devices may include user input buttons, switches, knobs, levers, keys, trackballs, touchpads, cameras, microphones, motion sensors, heat sensors, inertial sensors, touch sensors, or a combination of the above. Further, the interface(s) 118 may either be implemented as a Command Line Interface (CLI), a Graphical User Interface (GUI), a voice interface, or a web-based user-interface.

The memory 120 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other types of media/machine-readable medium suitable for storing electronic instructions. The memory 120 may include modules implemented as a program.

In accordance with various embodiments, several users may interact with the HIE system 102, using the user device 104. Although a single user device has been illustrated, several user devices could similarly be connected to the communication network 110. Further, each of the user devices may have a device ID. In various embodiments, the device ID may be a unique identification code such as an (International Mobile Equipment Identity) IMEI code or a product serial number. It should be noted that a user may use a single user device or multiple user devices. Further, multiple users may use a single user device or multiple user devices. Further, the one or more users may receive and/or provide healthcare related products and services. The one or more users may include, for example, patients, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but not limited to, a computer, a laptop, a tablet, a mobile phone, a smartphone, or a smart watch. In various embodiments, the user device 104 may include an imaging device that may be configured to capture a visual graphical element, the visual graphical element such as, but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. It should be noted that image data of the visual graphical element may be transmitted to the user device 104 via the communication network 110.

In accordance with various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In accordance with various embodiments, a group of databases 102b may be connected to the HIE system 102. In various embodiments, the group of databases 102b may be implemented over a blockchain network (such as a PTOYNet blockchain network or a PTOYNet Ethereum™ Blockchain network), and may be present as different databases installed at different locations. The group of databases 102b may include a subscriber database 132, a smart contracts database 134, an access rights database 136, and a patient database 138. The group of databases 102b may be configured to store data belonging to different users and data for functioning of the HIE system 102. Different databases can be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. For example, the data may represent the results of one medical test in a series of multiple medical tests.

In accordance with various embodiments, the group of databases 102b may operate collectively or individually. Further, the group of databases 102b may store data such as tables, objects, or other data structures. Further, the group of databases 102b may be configured to store data retrieved or processed by the HIE system 102. The data may include, but is not limited to, patient medical history, medical charts, medications, prescriptions, immunizations, test results, allergies, insurance provider(s), or billing information. Further, the data may be time-dependent and piece-wise. Further, the data may represent a subset of data for each patient. In an example, the data may represent results of a medical test in a series of multiple medical tests. Further, the data may be securely stored. In various embodiments, the data may be encrypted.

In accordance with various embodiments, information stored in the group of databases 102b may be accessed based on users' identities and/or the users' authorities. The users' identities may be verified in one or more ways such as, but not limited to, bio-authentication (or biometric authentication), password or PIN information, user device registrations, a second-level authentication, or a third-level authentication. For example, the users' identities may be verified by the HIE system 102. Information provided by the users in real-time may be used, by the HIE system 102, to confirm the users' identities. For example, the users' identities may be verified using a name, a password, one or more security questions, or a combination thereof. For example, a user may be identified using an encryption key and/or a decryption key.

In accordance with various embodiments, the data stored in the group of databases 102b may be accessed at different levels, for example using a first level subsystem and a second level subsystem. For example, a user may directly access the first level subsystem. To access data stored in the second level subsystem, the second level subsystem may be accessed through the first level subsystem. It should be noted that the communication between the first level subsystem and the second level subsystem may be encrypted. For example, the second level subsystem may be implemented over a blockchain network (such as a PTOYNet blockchain network or a PTOYNet Ethereum™ blockchain network). In one case, the PTOYNet blockchain network may be used to implement smart contracts.

In an exemplary scenario, a primary care physician may input data into the HIE system 102 using the user device 104. The data may be processed by the first level subsystem and the second level subsystem. This may be done successively. The data may be stored on the first level subsystem and/or the second level subsystem of the HIE system 102. This may be done successively. The data may include, but is not limited to, one or more instructions to a patient to see a physician specialist. Further, the data may be stored in one or more blockchains of the second level subsystem. The patient may be able to access the data relating to the patient's care provided by the primary care physician. This may be done successively. The patient may be able to retrieve the data using the user device 104 of the patient. This may be done successively.

In accordance with various embodiments, the patient may communicate with the physician specialist using the HIE system 102. It should be noted that the physician specialist may be able to access the data of the patient from the first level subsystem and/or the second level subsystem. Further, the physician specialist may be able to communicate with the patient. It should be noted that all (or substantially all) communications between the primary care physician, the physician specialist, and the patient may be stored in and may be accessible from a blockchain network.

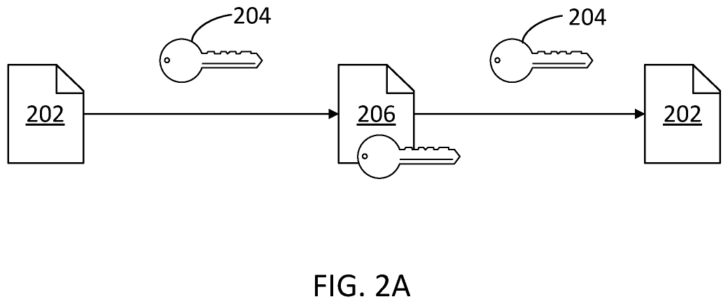

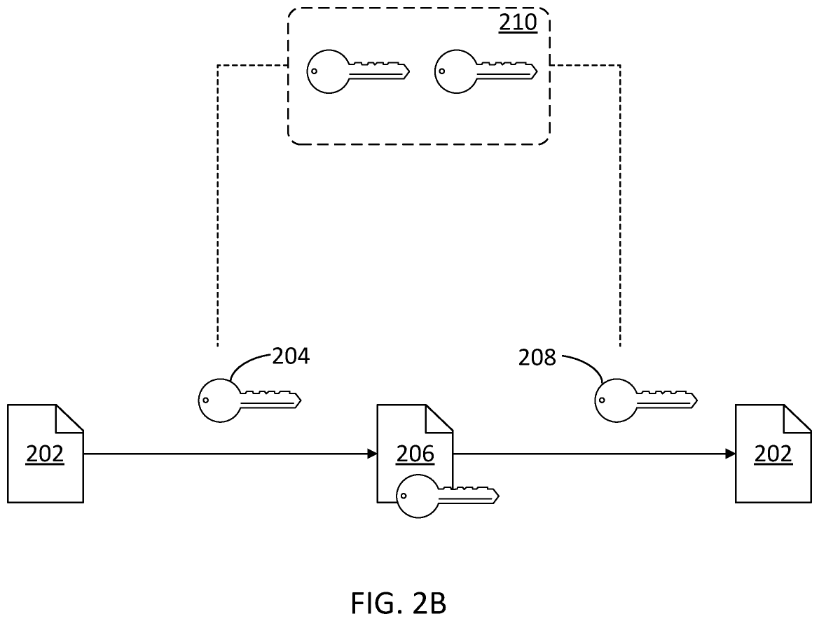

FIG. 2A illustrates a method for symmetric encryption of data, in accordance with various embodiments. Original data 202 may be encrypted using a key 204 to obtain an encrypted data 206. The encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2B illustrates a method for asymmetric encryption of data, in accordance with various embodiments. Original data 202 may be encrypted using a key 204 to obtain encrypted data 206. The encrypted data 206 may be decrypted using another key 208 to obtain the original data 202. It should be noted that encryption and decryption of the data may be performed using different keys, e.g., a key pair 210.

In some embodiments, the steps illustrated in FIGS. 2A-B may be initiated by users who generate a new profile on the blockchain network. Private keys may be stored in decentralized and distributed hashes through the blockchain network. In some embodiments, the steps illustrated in FIGS. 2A-B may be partially performed in either one of devices 104 and 106, in HIE system 102, or financial platform 108. For example, in some embodiments, HIE system 102 may install a software development kit (SDK) or a key generator application in user device 104 or in third party device 106 to perform at least some of the steps illustrated in FIGS. 2A-B. Likewise, keys 204, 208, and key pair 210 may be stored in a memory of either one of devices 104, 106, in HIE system 102, or in financial platform 108, or in an associated database (e.g., any one of databases 102b).

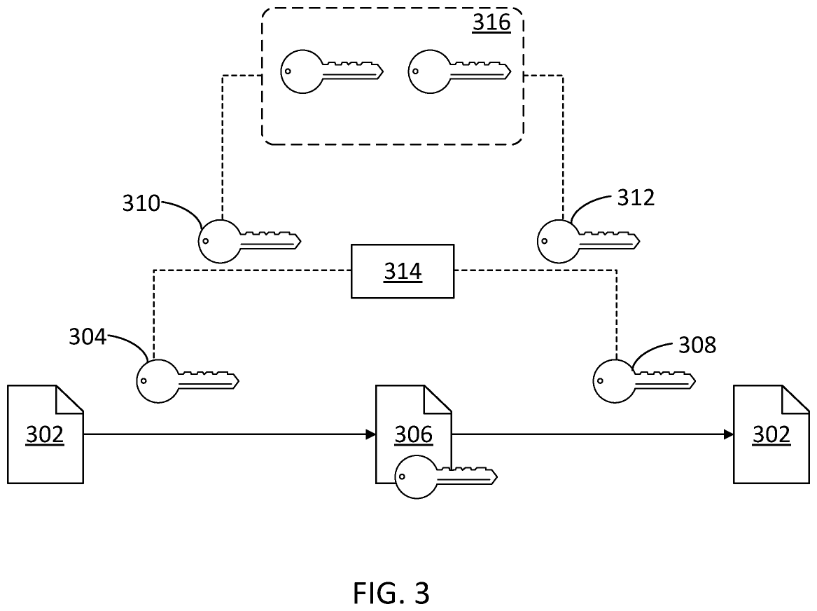

FIG. 3 illustrates a method for hybrid encryption of data, in accordance with various embodiments. Both symmetric encryption and asymmetric encryption techniques may be used in tandem. For example, the symmetric encryption technique may be used to encrypt data 302 using a symmetric key 304 for producing encrypted data 306. The encrypted data 306 may be decrypted using another symmetric key 308 for obtaining data 302. Further, a public key 310 may be used to encrypt the symmetric key 304 and a private key 312 may be used to encrypt the symmetric key 308, stored as an encrypted key 314. The public key 310 and the private key 312 may form a key pair 316.

In some embodiments, the steps illustrated in FIG. 3 may be initiated by users who generate a new profile on the blockchain network. Private keys may be stored in decentralized and distributed hashes through the blockchain network. In some embodiments, the steps illustrated in FIG. 3 may be partially performed in either one of devices 104 and 106, in HIE system 102, or financial platform 108. For example, in some embodiments, HIE system 102 may install a software development kit (SDK) or a key generator application in user device 104 or in third party device 106 to perform at least some of the steps illustrated in FIG. 3. Likewise, keys 204, 208, and key pair 210 may be stored in a memory of either one of devices 104, 106, in HIE system 102, or in financial platform 108, or in an associated database (e.g., any one of databases 102b).

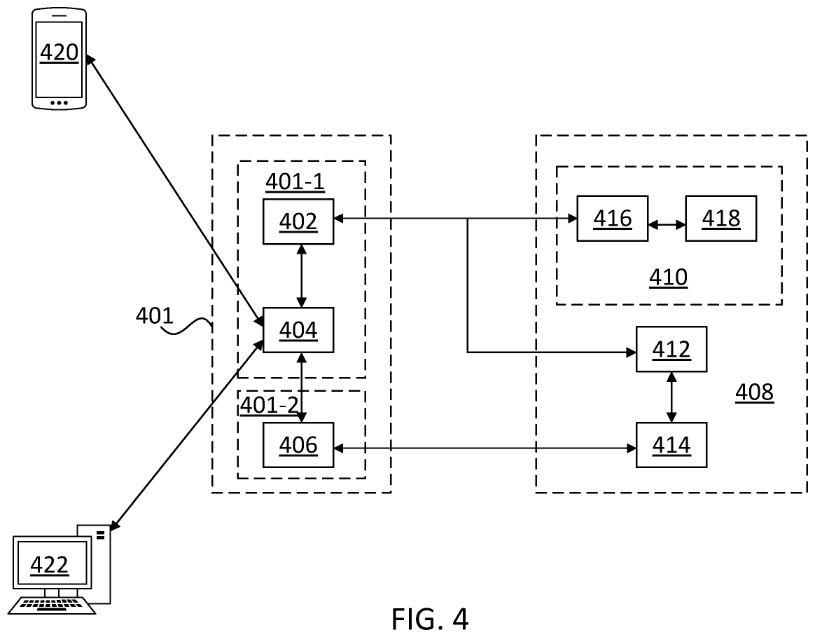

FIG. 4 illustrates an example of a system 401 for storing and accessing data in a health care network, according to some embodiments. A first level subsystem 401-1 may include a core service component 402 and a Remote Procedure Call (RPC) component 404. A second level subsystem 401-2 may include a blockchain node 406. Blockchain node 406 may be a public node or a private node in a blockchain network having a layer over a public blockchain network, enabling the private node to perform private transactions via consensus algorithms (e.g., a Quorum blockchain node). In various embodiments, first level subsystem 401-1 may include core service component 402, and second level subsystem 401-2 may include RPC component 404 and the blockchain node 406. Further, the core service component 402 of the first level subsystem may be present in communication with third-party servers and databases of a hospital computing network 408. The hospital computing network 408 may include a file system module 410, an EHR synchronization service 412, and a blockchain node 414 (e.g., a Quorum blockchain node). Further, the file system module 410 may include a file system manager 416 and a file system node 418. The blockchain node 406 of the second level subsystem 401-2 may communicate with the blockchain node 414 of the hospital computing network 408. Patients may access the health care network for storing data through a user device 420, and a representative of a hospital may access the health care network through another user device 422.

In accordance with various embodiments, the representative of the hospital may want to synchronize Electronic Health Record (EHR) data of a patient, e.g., by using corresponding blockchain hashes. First level subsystem 401-1 and second level subsystem 401-2 may ask the patient for permission to allow a representative of the hospital to store the EHR data of the patient, through the file system module 410. This may be done successively. Based at least on the permission granted by the patient, a signed transaction may be created to confirm the permission of the hospital to store the EHR data. Further, the signed transaction may activate a smart contract that may add hospital identification information such as a blockchain address to a list of permitted users. In some embodiments, the signed transaction and the smart contract are stored in file system module 410.

In accordance with various embodiments, the signed transaction may be transmitted from the user device to the RPC component 404 of the first level subsystem 401-1 and/or the second level subsystem 401-2. The RPC component 404 may communicate the signed transaction to the blockchain node 406 of the second level subsystem. The blockchain node 406 may activate one or more smart contracts. The blockchain node 406 may revise a state of one or more blockchains.

In accordance with various embodiments, based at least on the permission granted by the patient, the EHR synchronization service may obtain a patient, or a list of patients, from the RPC component 404. The EHR synchronization service may confirm whether the patient has granted permission. Based at least on the permission, the first level subsystem and the second level subsystem may obtain the EHR data and may calculate a hash function for the EHR data. The HIE system 102 may match the hash function of the EHR data with a hash function for the patient blockchain on the blockchain node 406 of the second level subsystem. In various embodiments, if the hash function of the EHR data matches with the hash function for the patient blockchain on the blockchain node 406 of the second level subsystem, the EHR data of the patient may remain unchanged.

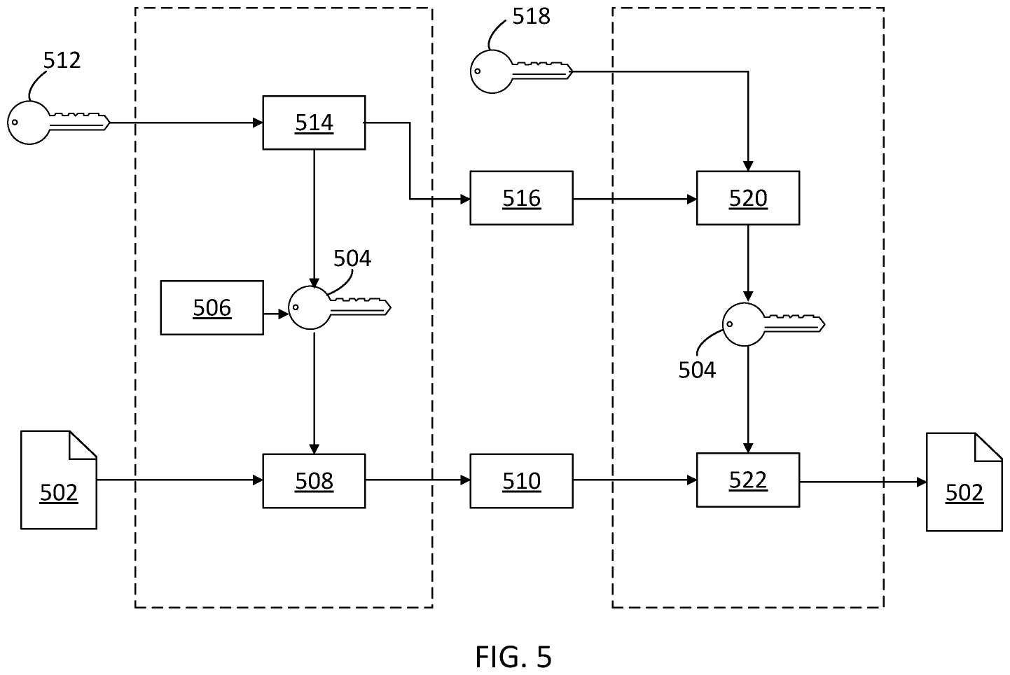

FIG. 5 illustrates an example of a system for storing and accessing data in a health care network implemented specifically over a blockchain network as disclosed herein, according to some embodiments (cf. FIGS. 1 and 4). The HIE system 102 may execute an application for determining permission from the user for obtaining EHR data 502. In one case, if the user grants the permission, the HIE system 102 may obtain the EHR data 502 for calculating a hash function for the EHR data 502. Further, the HIE system 102 may match the hash function of the EHR data 502 with a hash function for the user blockchain on the blockchain node of the second level sub-system. In one case, if the two hash functions match, there is no change to the user's EHR data 502. In various embodiments, if the two hash functions do not match, the HIE system 102 may generate a random string (e.g., secret key 504) through a random key generator 506. The secret key 504 may be used for Advanced Encryption Standard (AES) encryption of the EHR data 502, in an AES encryptor 508, for generating encrypted EHR data 510.

In accordance with various embodiments, secret key 504 may then be encrypted by, for example, a Rivest-Shamir-Adleman (RSA) public key 512 of the patient, in an RSA encryptor 514, to generate an encrypted secret key 516. The HIE system 102 may further send the encrypted EHR data 510 to the core service component 402 for forwarding the data to the file system manager 416 of the hospital computing network 408 for storage. Further, the file system manager 416 may send a file system hash function to the core service component 402 for further sending the file system hash function to EHR synchronization service 412. The EHR synchronization service 412 may further update the patient smart contract with the new file system hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to an RPC component 404 for granting permission to the hospital representative to view the EHR data 502. Once the permission is granted, the signed transaction may be added to the blockchain node 414 and a new smart contract will be created for a blockchain corresponding to the hospital representative. After adding the signed transaction, the hospital representative may be able to view the EHR data 502 of the user on a device.

In accordance with various embodiments, in order to view the EHR data 502 on the device, the HIE system 102 may collect the encrypted EHR data 510 from the user's blockchain and may decrypt the encrypted EHR data 510 using a patient's RSA private key 518. The HIE system 102 may decrypt the encrypted secret key 516, in an RSA decryptor 520, using an RSA private key of the hospital representative. The encrypted EHR data 510 may be decrypted using the RSA public key 512 of the hospital representative, in an AES decryptor 522. Further, the HIE system 102 may load the decrypted EHR data 502 to the smart contract previously created for the hospital representative.

Post loading, the RPC component 404 may obtain the signed transaction from the patient's user device and transmit the signed transaction to the blockchain node 406 of the second level subsystem. The blockchain node 406 may confirm ownership of the signed transaction and may execute the smart contract for the hospital representative to view the user's data.

In accordance with various embodiments, the patient may decline permission for the hospital representative to have access to the EHR data 502. In such a scenario, the user, through a user device, may send a signed transaction revoking permission to the RPC component 404. The RPC component 404 may forward the signed transaction to the blockchain node 406 of the second level subsystem. The blockchain node 406 may confirm ownership of the signed transaction and may delete the smart contract previously created to allow the hospital representative to have access to the patient's EHR data 502.

In accordance with various embodiments, the subscriber database 132 may be configured to store information related to the third party user or the subscriber. It should be noted that the subscriber database 132 may be used to determine whether the third party user exists in the HIE system 102.

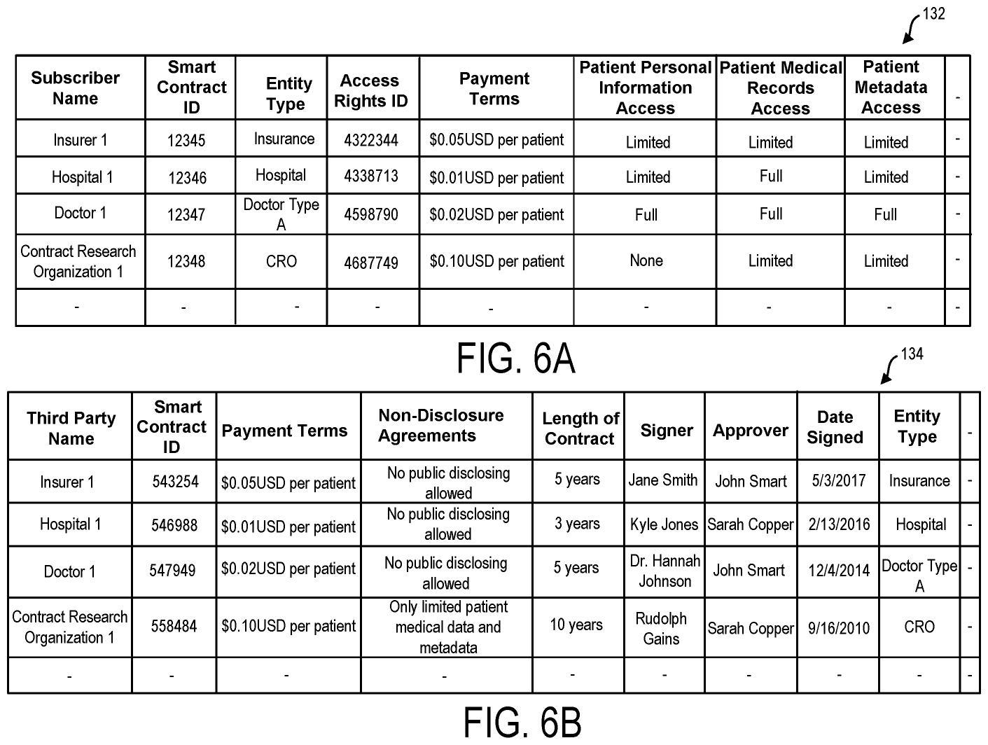

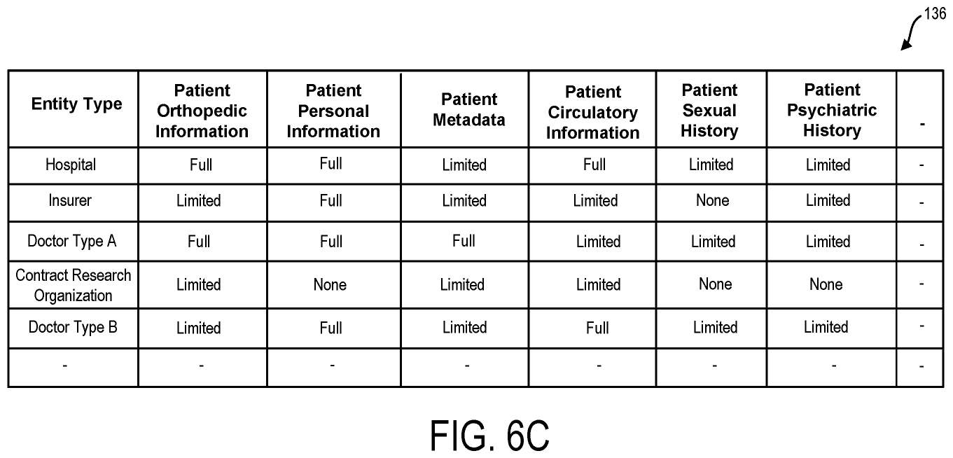

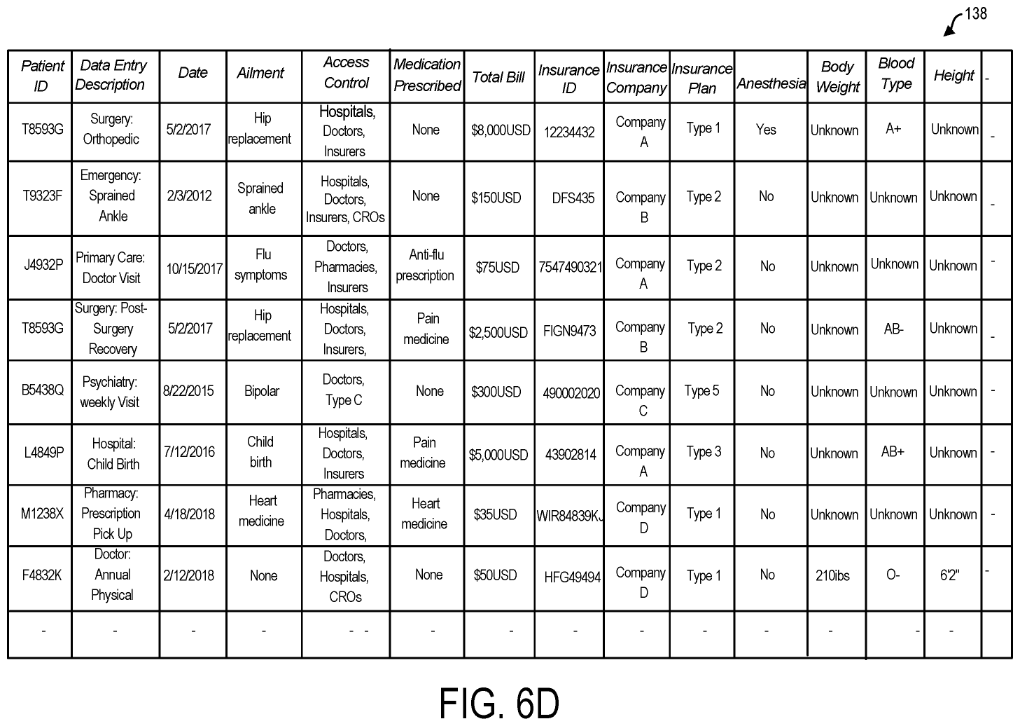

FIG. 6A illustrates the information stored in subscriber database 132, according to some embodiments. Accordingly, the information may include, without limitation, a name of a third party user, a smart contract ID, a type of an entity, an access rights ID, payment terms, and/or a level of access to one or more fields of patient data. The one or more fields may be, for example, patient personal information, patient medical records, or patient metadata. For example, a type of the entity may be an insurance company or a hospital. The payment terms may be, for example, 0.001 cryptocurrency per patient. In accordance with various embodiments, the smart contracts database 134 may be configured to store verified potential third party user's smart contracts. The smart contracts database 134 may be accessed to determine if the third party user is trying to request the patient information.

FIG. 6B illustrates the smart contracts database 134 storing information, according to some embodiments. The information may include, without limitation, a third party name, a smart contract ID, payment terms, a Non-Disclosure Agreements (NDA), a length of contract, a signer, an approver, a signature date, or an entity type. A smart contract as disclosed herein includes an algorithm that is executed when conditions for parameters defining the terms of the contract are met.

FIG. 6C illustrates a configuration of the access rights database 136 to store one or more types of patient information, according to some embodiments. Further, the access rights database 136 may be accessed to determine what information to show to the third party user and what information to restrict. As shown in FIG. 6C, the one or more types of the patient information may include, but is not limited to, patient orthopedic information, patient personal information, patient metadata, patient circulatory information, patient sexual history, or patient psychiatric history. Further, the access rights database 136 may store, for example, an entity type and a level of access for each type of the patient information. The level of access may be “Full,” “Limited,” or “None,” depending on the type of entity accessing the information.

FIG. 6D illustrates a patient information stored in patient database 138, according to various embodiments. The patient database 138 may store multiple types of data attached to a name of the patient. Patient database 138 may include, but is not limited to, a patient ID, a data entry description, date, ailment, access control, medication prescribed, total bill, insurance ID, insurance company, insurance plan, or anesthesia. Further, patient database 138 may store one or more parameters of the patient such as, for example, body weight, blood type, or height.

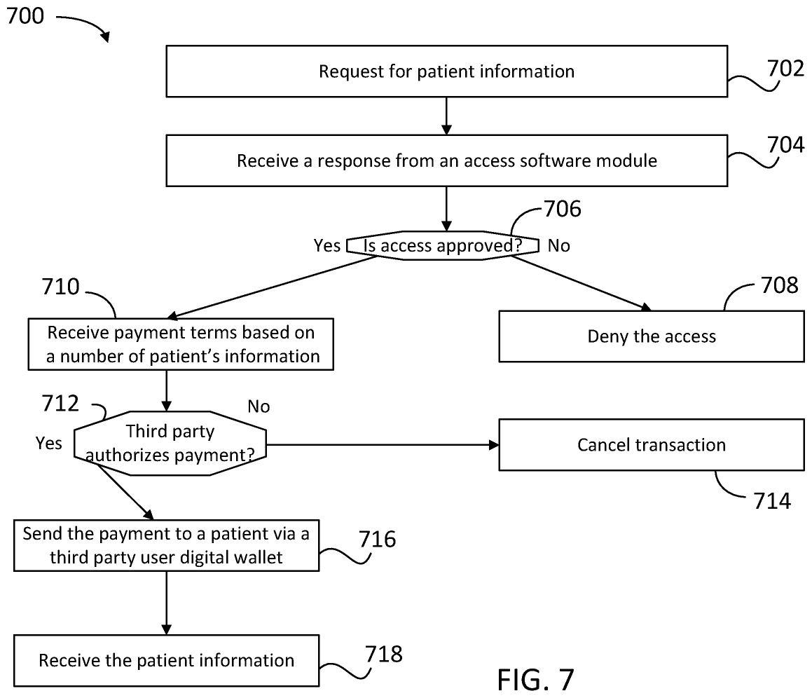

FIG. 7 illustrates an example of a flowchart 700 showing a method that can be performed using a third party interface on a third party device 106, according to some embodiments. A process carried out using the third party interface on the third party device 106 will now be explained with reference to flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to the example flowchart of FIG. 7, the third party device 106 may place a request for obtaining patient information, at step 702. The request may be sent to the access software module 122. The third party device 106 may receive a response from the access software module 122, at step 704. The third party device 106 may determine whether an access is approved from a patient, at step 706. In various embodiments, if the access is not approved, the third party device 106 may be informed about denial of the access, at step 708. In various embodiments, if the access is approved, the third party device 106 may receive payment terms based on a number of patient's information requested, at step 710. It may be determined if the third party user authorizes the making of a payment, at step 712. In various embodiments, if the payment is not authorized by the third party user, then the third party device 106 may inform about cancellation of the transaction, at step 714. In various embodiments, if the payment is authorized by the third party user, the third party device 106 may send the payment to the patient via the third party user digital wallet 114, at step 716. In various embodiments, the payment may be sent to the HIE system 102. Thereafter, the third party device 106 may receive the patient information, at step 718. It should be noted that the patient information may be encrypted and the third party device 106 may receive public patient ID key(s) to access the patient information stored at the blockchain database. Upon receiving the public patient ID key(s), the information request may be processed and the information may be sent to the third party user.

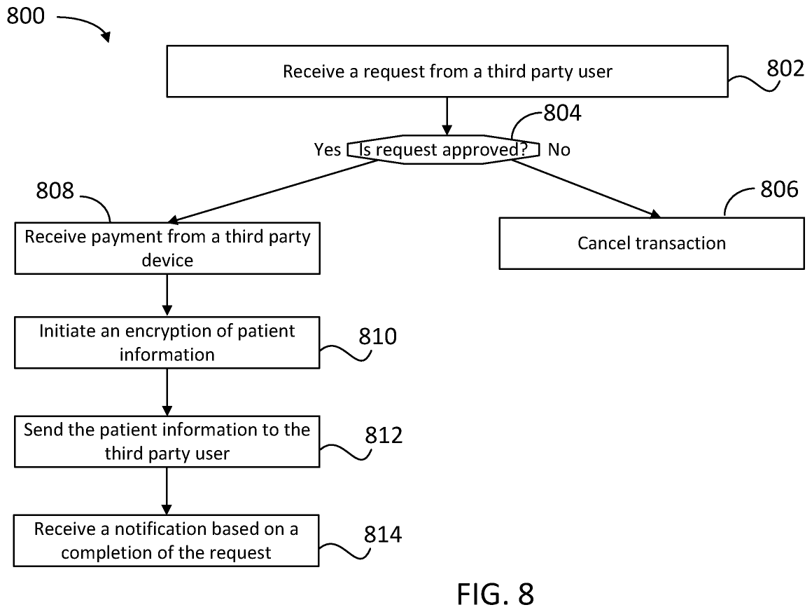

FIG. 8 illustrates an example of a flowchart 800 showing a method for using a patient interface present on the user device 104, according to some embodiments. A process carried out using the patient interface present on the user device 104 will now be explained with reference to example flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the user device 104 may receive a request from a third party user to access the patient information, at step 802. The request may be received from the third party device 106 via the HIE system 102. The user device 104 may check whether the patient has approved the request, at step 804. In various embodiments, if the request is not approved, then the user device 104 may inform about the cancellation of the transaction, at step 806. In various embodiments, if the request is approved, then the user device 104 may receive the payment for the patient from the third party device 106, at step 808. The payment may be received in the user digital wallet 112 via the financial platform 108. After receiving the payment, the user device 104 may initiate encryption of the patient information, at step 810. The user device 104 may send the patient information to the third party user, at step 812. Thereafter, the user device 104 may receive a notification based on a completion of the request, at step 814. The notification may include information that the patient information is no longer being shared and the pubic key and the private key have been negated.

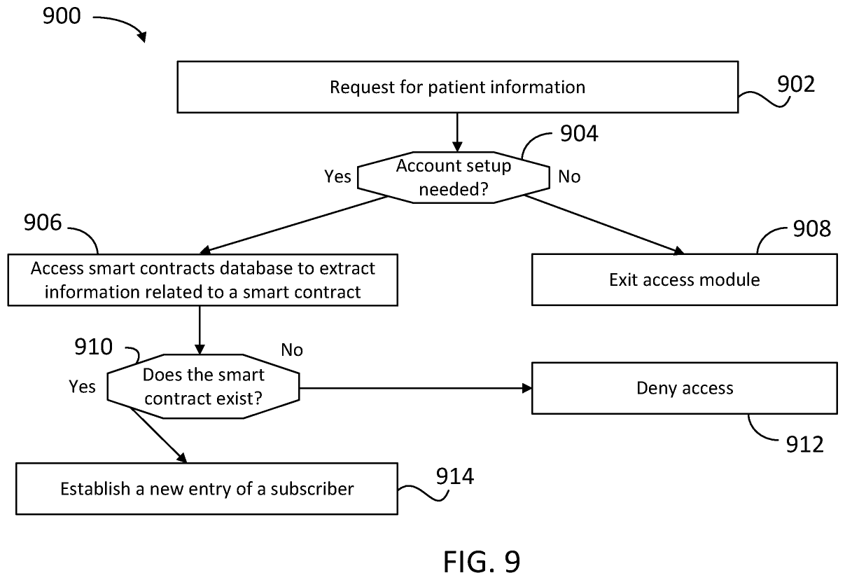

FIG. 9 illustrates an example flowchart 900 for operating the setup module 124, according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the setup module 124 may receive information related to a third party user, at step 902. The information may include, but not limited to, a name of the third party user or an identification of the third party user. The setup module 124 may determine whether an account setup of the third party user is needed, at step 904. In various embodiments, if the account setup is needed, the setup module 124 may access the smart contracts database 134 to extract information related to a smart contract, at step 906. In various embodiments, if the account setup is not needed, then the access module 126 may be exited, at step 908. The smart contract may include payment terms or a type of entity. The setup module 124 may determine whether the smart contract of the third party user exists, at step 910. In various embodiments, if the smart contract does not exist, the setup module 124 may deny the access, at step 912. It should be noted that the third party user may have to establish a smart contract first outside of the HIE system 102. In various embodiments, if the smart contract exists, then the setup module 124 may establish a new entry of a subscriber, at step 914. The subscriber may refer to a new third party user. The new entry of the subscriber may be established by combining information extracted from the smart contracts database 134 and information extracted from the access rights database 136.

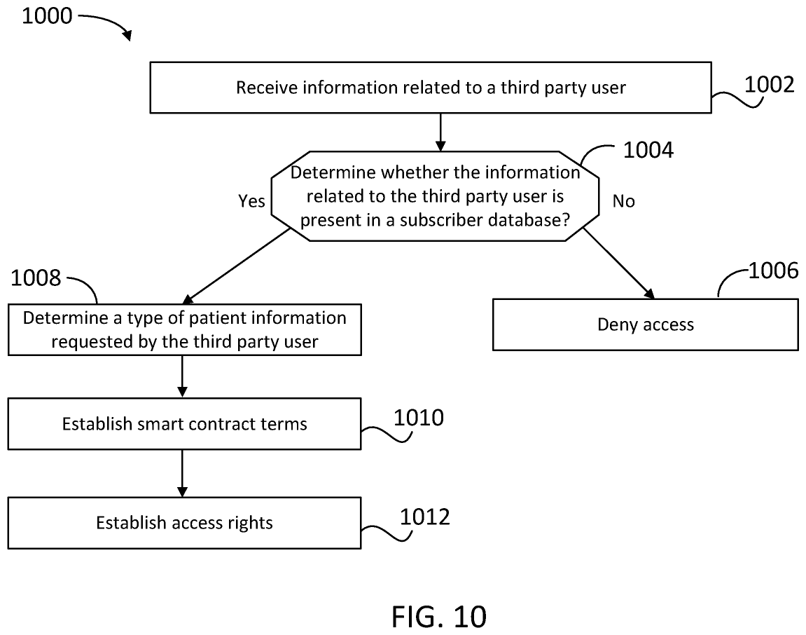

FIG. 10 illustrates a flowchart 1000 for operating access module 126, according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the access module 126 may receive the information related to the third party user, at step 1002. The information may include, but is not limited to, a name of the third party user or an identification of the third party user. For example, the information may be specific patient information, a large number of patients' information, or pharmaceutical information. The access module 126 may determine whether the information related to the third party user is present in the subscriber database 132, at step 1004. In various embodiments, if the information related to the third party user is not present in the subscriber database 132, the access module 126 may deny the access, at step 1006. In various embodiments, if the information related to the third party user is present in the subscriber database 132, the access module 126 may determine a type of the patient information requested by the third party user, at step 1008.

The access module 126 may establish smart contract terms, at step 1010. The smart contract terms may include information such as, for example, an entity type or other restrictions on the patient information. In various embodiments, the entity type may be an insurance company or a hospital. Thereafter, the access module 126 may establish access rights, at step 1012. The access rights may allow the third party user to view the information that is allowed. For example, if a Contract Research Organization (CRO) wishes to perform a search on patients using a drug, the access module 126 may provide access rights to information such as patient metadata or patient pharmaceutical data.

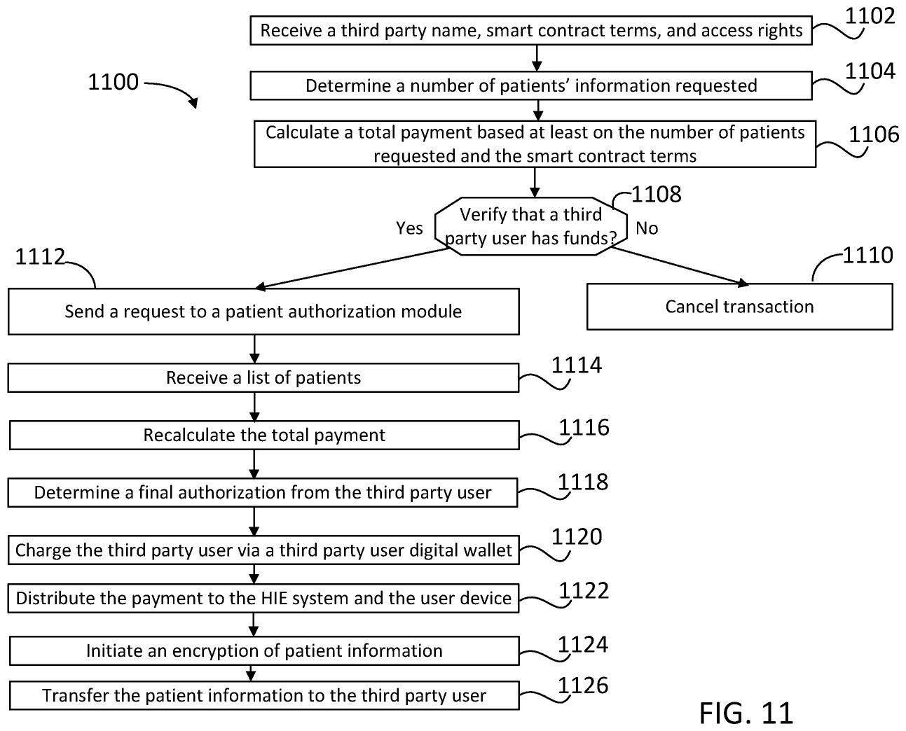

FIG. 11 illustrates an example of a flowchart 1100 showing a method performed by a payment module 128, according to various embodiments. Functioning of the payment module 128 will now be explained with reference to flowchart 1100 shown in FIG. 11. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the payment module 128 may receive, for example, the third party name, the smart contract terms, and the access rights from the access module 126, at step 1102. Successively, the payment module 128 may determine a number of patients' information requested, at step 1104. In various embodiments, the payment module 128 may determine a volume of network usage required. The payment module 128 may calculate a total payment based at least on the number of patients requested and the smart contract terms, at step 1106. The smart contract terms may be accessed from the smart contracts database 134. For example, a hospital may pay 0.01 USD per patient in order to access the patient's information. For example, an insurance company may pay 0.05 USD per patient in order to access the patient's information.

Payment module 128 may verify that the third party user has sufficient funds, at step 1108. In various embodiments, if the third party user does not have sufficient funds, the payment module 128 may cancel transaction, at step 1110. In various embodiments, if the third party user has sufficient funds, the payment module 128 may send a request to the patient authorization module 130, at step 1112. The request may correspond to an authorization request for accessing the patient information.

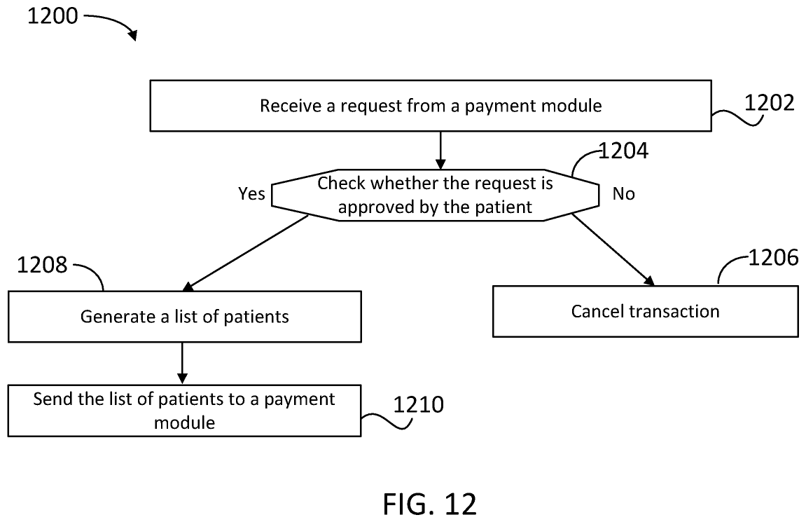

Returning to flowchart 1100 of FIG. 11, the payment module 128 may receive the list of patients from the patient authorization module 130, at step 1114, via a process such as the example process discussed with reference to the flow chart 1200 of FIG. 12. The payment module 128 may recalculate the total payment, at step 1116. The total payment may be recalculated based at least on the number of patients that have approved the request. The payment module 128 may determine a final authorization from the third party user, at step 1118. The transaction may be cancelled when the final authorization is not received from the third party. If the final authorization is received from the third party user, the payment module 128 may charge the third party user via the third party user digital wallet 114, at step 1120. It should be noted that the payment may be transferred via the financial platform 108. The payment module 128 may distribute the payment to the HIE system 102 and the user device 104, at step 1122. The payment may be sent, for example, to the user device 104 via the user digital wallet 112. The payment module 128 may initiate an encryption of the patient information, at step 1124. It should be noted that the encryption may be performed using a public key for the third party user and a private key for the patient. In various embodiments, the patient information may be retrieved from the patient database 138. Successively, the payment module 128 may transfer the patient information to the third party user, at step 1126. It should be noted that the payment module 128 may send the public key to the third party user along with the patient information. Thereafter, the public key and private key may be cancelled.

FIG. 12 illustrates a flowchart 1200 for operating patient authorization module 130, according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the patient authorization module 130 may receive the request from the payment module 128, at step 1202. Successively, the patient authorization module 130 may check that the request is approved by the patient, at step 1204. In various embodiments, if the request is not approved, the patient authorization module 130 may cancel the transaction, at step 1206. In various embodiments, if the request is approved, then the patient authorization module 130 may generate a list of the patients who approved the request, at step 1208. Thereafter, the patient authorization module 130 may send the list of the patients to the payment module 128, at step 1210.

### Computer System

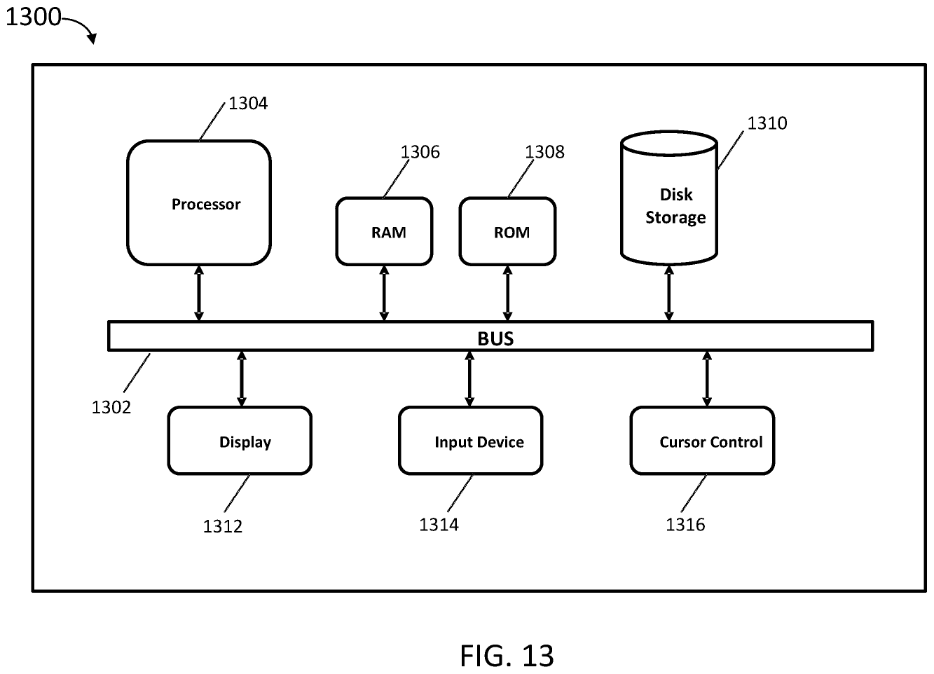

FIG. 13 is a block diagram that illustrates a computer system 1300, upon which embodiments, or portions of the embodiments, of the present teachings may be implemented. In various embodiments of the present teachings, computer system 1300 can include a bus 1302 or other communication mechanism for communicating information, and a processor 1304 coupled with bus 1302 for processing information. In various embodiments, computer system 1300 can also include a memory 1306, which can be a random access memory (RAM) or other dynamic storage device, coupled to bus 1302 for determining instructions to be executed by processor 1304. Memory 1306 also can be used for storing temporary variables or other intermediate information during execution of instructions to be executed by processor 1304. In various embodiments, computer system 1300 can further include a read-only memory (ROM) 1308 or other static storage device coupled to bus 1302 for storing static information and instructions for processor 1304. A storage device 1310, such as a magnetic disk or optical disk, can be provided and coupled to bus 1302 for storing information and instructions.

In various embodiments, computer system 1300 can be coupled via bus 1302 to a display 1312, such as a cathode ray tube (CRT) or liquid crystal display (LCD), for displaying information to a computer user. An input device 1314, including alphanumeric and other keys, can be coupled to bus 1302 for communicating information and command selections to processor 1304. Another type of user input device is a cursor control 1316, such as a mouse, a trackball, or cursor direction keys for communicating direction information and command selections to processor 1304 and for controlling cursor movement on display 1312. This input device 1314 typically has two degrees of freedom in two axes, a first axis (e.g., x) and a second axis (e.g., y), that allows the device to specify positions in a plane. However, it should be understood that input devices 1314 allowing for 3-dimensional (x, y, and z) cursor movement are also contemplated herein.

Consistent with certain implementations of the present teachings, results can be provided by computer system 1300 in response to processor 1304 executing one or more sequences of one or more instructions contained in memory 1306. Such instructions can be read into memory 1306 from another computer-readable medium or computer-readable storage medium, such as storage device 1310. Execution of the sequences of instructions contained in memory 1306 can cause processor 1304 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1304 for execution. Such a medium can take many forms, including but not limited to, non-volatile media, volatile media, and transmission media. Examples of non-volatile media can include, but are not limited to, optical, solid state, and magnetic disks, such as storage device 1310. Examples of volatile media can include, but are not limited to, dynamic memory, such as memory 1306. Examples of transmission media can include, but are not limited to, coaxial cables, copper wire, and fiber optics, including the wires that include bus 1302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to a computer-readable medium, instructions or data can be provided as signals on transmission media included in a communications apparatus or system to provide sequences of one or more instructions to processor 1304 of computer system 1300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein including flow charts, diagrams, and the accompanying disclosure can be implemented using computer system 1300 as a standalone device or on a distributed network of shared computer processing resources such as a cloud computing network.

In accordance with various embodiments, the systems and methods described herein can be implemented using computer system 1300 as a standalone device or on a distributed network of shared computer processing resources such as a cloud computing network. As such, a non-transitory computer-readable medium can be provided in which a program is stored for causing a computer to perform the disclosed methods for identifying mutually incompatible gene pairs.

It should also be understood that the preceding embodiments can be provided, in whole or in part, as a system of components integrated to perform the methods described. For example, in accordance with various embodiments, the methods described herein can be provided as a system of components or stations for analytically determining novelty responses.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, their specific configuration, communication, and physical orientation with respect to each other. One skilled in the art should readily appreciate that these components can have various configurations and physical orientations (e.g., wholly separate components, units, and subunits of groups of components, different communication regimes between components).

Embodiments as disclosed herein include:

A. A computer-implemented method for managing payments for accessing patients' information includes receiving, from a third party user, a request to access a patient's information stored in a blockchain database and determining a type of the patients' information. The computer-implemented method also includes receiving an authorization from a patient to provide an access of the patients' information to the third party user, calculating, based at least on the type of the patients' information and the authorization from the patient, a payment value to be paid by the third party user for the patient's information, and allowing the access to the patients' information to the third party user upon receipt of the payment value.

B. A system for managing payments for accessing patients' information includes a memory storing instructions and one or more processors configured to execute the instructions. The instructions cause the system to receive, from a third party user, a request to access a patient's information stored in a blockchain database, to determine a type of the patients' information, and to receive an authorization from a patient to provide an access of the patients' information to the third party user. The instructions also cause the system to calculate, based at least on the type of the patients' information and the authorization from the patient, a payment value to be paid by the third party user for the patient's information, and to allow the access to the patients' information to the third party user upon receipt of the payment value.

C. A computer-implemented method for accessing a patient information in a health information exchange includes requesting, via a communication network, the patient information from the health information exchange. The computer-implemented method also includes receiving, from a server in the health information exchange, a condition for access when an access to the patient information is approved, authorizing a transaction to access the patient information when the condition is fulfilled, and receiving an address in a blockchain database, and a key to access a document in the address, wherein the document includes at least a portion of the patient information as determined by the condition for access.

Each of embodiments A, B, and C may have one or more of the following additional elements in any combination: Element 1, wherein the third party user is an individual belonging to a hospital, insurance company, pharmaceutical company, or a Contract Research Organization, further including providing an access right to the patient information based on the type of the patients' information and the third party user. Element 2, wherein the type of the patients' information is selected from a group consisting of a patient orthopedic information, a patient personal information, a patient metadata, a patient circulatory information, a patient sexual history, and a patient psychiatric history, and wherein allowing access to the patient's information includes verifying an access right based on a contract term associating the type of the patients' information and a type of third party user with the access right. Element 3, further including receiving, from the third party user, an authorization to pay the payment value based on a payment term. Element 4, wherein allowing the third party user to access the patient's information includes encrypting the patient's information in the blockchain database and transmitting a public key to the third party user, the public key configured to unlock the blockchain database. Element 5, wherein the authorization from the patient includes a signed transaction, further including adding the signed transaction to a quorum blockchain node and creating a new contract for a blockchain associated with the third party user. Element 6, wherein calculating a payment value to be paid by the third party user includes determining that a third party user information is present in a subscriber database and selecting a contract term based on the third party user information and the type of the patients' information. Element 7, further including applying a hash function to an electronic health record for the patient based on the authorization from the patient, and matching the hash function with a hash function for a patient blockchain in the blockchain database. Element 8, further including generating a random string for a secret key to encrypt the patient information when a hash function for the patient's information does not match with a hash function for a patient blockchain in the blockchain database. Element 9, wherein the patient's information is stored in a hospital server under a secret encryption key, further including updating a patient blockchain with the secret encryption key.

Each of embodiments A, B, and C may also have one or more of the following additional elements in any combination: Element 10, wherein the third party user is an individual belonging to a hospital, insurance company, pharmaceutical company, or a Contract Research Organization, the one or more processors further configured to provide an access right to the patient information based on the type of the patients' information and the third party user. Element 11, wherein the type of the patients' information is selected from a group consisting of a patient orthopedic information, a patient personal information, a patient metadata, a patient circulatory information, a patient sexual history, and a patient psychiatric history, and wherein to allow access to the patient's information the one or more processors execute instructions to verify an access right based on a contract term associating the type of the patients' information and a type of third party user with the access right. Element 12, wherein the one or more processors further execute instructions to receive, from the third party user, an authorization to pay the payment value based on a payment term. Element 13, wherein to allow the third party user to access the patient's information the one or more processors execute instructions to encrypt the patient's information in the blockchain database and transmitting a public key to the third party user, the public key configured to unlock the blockchain database.

Each of embodiments A, B, and C may also have one or more of the following additional elements in any combination: Element 14, wherein the condition for access is a payment term based on a type of patient information requested and on an identity of a third party making the request, further including providing, to the server in the health information exchange, a credential indicative of the identity of the third party. Element 15, further including decrypting the portion of the patient information using the key and displaying the portion of the patient information in a third party user device. Element 16, wherein the key includes a private key and a random secret key, further including finding a hashing function to decrypt the portion of the patient information based on the private key and the random secret key. Element 17, wherein the patient information includes multiple patients, further including receiving, from the server in the health information exchange, a list of the patients satisfying a condition in the request, and selecting one or more of the patients from the list, based on the condition.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for managing payments for accessing patients' information, the method comprising:
receiving, from a third party user, a request to access a patient's information stored in a blockchain database;
determining a type of the patients' information;
receiving an authorization from a patient to provide an access of the patients' information to the third party user;
calculating, based at least on the type of the patients' information and the authorization from the patient, a payment value to be paid by the third party user for the patient's information; and
allowing the access to the patients' information to the third party user upon receipt of the payment value.

2. The computer-implemented method of claim 1, wherein the third party user is an individual belonging to a hospital, insurance company, pharmaceutical company, or a Contract Research Organization, further comprising providing an access right to the patient information based on the type of the patients' information and the third party user.

3. The computer-implemented method of any one of claims 1 and 2, wherein the type of the patients' information is selected from a group consisting of a patient orthopedic information, a patient personal information, a patient metadata, a patient circulatory information, a patient sexual history, and a patient psychiatric history, and wherein allowing access to the patient's information comprises verifying an access right based on a contract term associating the type of the patients' information and a type of third party user with the access right.

4. The computer-implemented method of any one of claims 1 through 3, further comprising receiving, from the third party user, an authorization to pay the payment value based on a payment term.

5. The computer-implemented method of any one of claims 1 through 4, wherein allowing the third party user to access the patient's information comprises encrypting the patient's information in the blockchain database and transmitting a public key to the third party user, the public key configured to unlock the blockchain database.

6. The computer-implemented method of any one of claims 1 through 5, wherein the authorization from the patient includes a signed transaction, further comprising adding the signed transaction to a quorum blockchain node and creating a new contract for a blockchain associated with the third party user.

7. The computer-implemented method of any one of claims 1 through 6, wherein calculating a payment value to be paid by the third party user comprises determining that a third party user information is present in a subscriber database and selecting a contract term based on the third party user information and the type of the patients' information.

8. The computer-implemented method of any one of claims 1 through 7, further comprising applying a hash function to an electronic health record for the patient based on the authorization from the patient, and matching the hash function with a hash function for a patient blockchain in the blockchain database.

9. The computer-implemented method of any one of claims 1 through 8, further comprising generating a random string for a secret key to encrypt the patient information when a hash function for the patient's information does not match with a hash function for a patient blockchain in the blockchain database.

10. The computer-implemented method of any one of claims 1 through 9, wherein the patient's information is stored in a hospital server under a secret encryption key, further comprising updating a patient blockchain with the secret encryption key.

11. A system for managing payments for accessing patients' information, the system comprising:
a memory storing instructions; and
one or more processors configured to execute the instructions and cause the system to:
receive, from a third party user, a request to access a patient's information stored in a blockchain database;
determine a type of the patients' information;
receive an authorization from a patient to provide an access of the patients' information to the third party user;
calculate, based at least on the type of the patients' information and the authorization from the patient, a payment value to be paid by the third party user for the patient's information; and
allow the access to the patients' information to the third party user upon receipt of the payment value.

12. The system of claim 11, wherein the third party user is an individual belonging to a hospital, insurance company, pharmaceutical company, or a Contract Research Organization, the one or more processors further configured to provide an access right to the patient information based on the type of the patients' information and the third party user.

13. The system of any one of claims 11 and 12, wherein the type of the patients' information is selected from a group consisting of a patient orthopedic information, a patient personal information, a patient metadata, a patient circulatory information, a patient sexual history, and a patient psychiatric history, and wherein to allow access to the patient's information the one or more processors execute instructions to verify an access right based on a contract term associating the type of the patients' information and a type of third party user with the access right.

14. The system of any one of claims 11 through 13, wherein the one or more processors further execute instructions to receive, from the third party user, an authorization to pay the payment value based on a payment term.

15. The system of any one of claims 11 through 14, wherein to allow the third party user to access the patient's information the one or more processors execute instructions to encrypt the patient's information in the blockchain database and transmitting a public key to the third party user, the public key configured to unlock the blockchain database.

16. A computer-implemented method for accessing a patient information in a health information exchange, the method comprising:
requesting, via a communication network, the patient information from the health information exchange;
receiving, from a server in the health information exchange, a condition for access when an access to the patient information is approved;
authorizing a transaction to access the patient information when the condition is fulfilled; and
receiving an address in a blockchain database, and a key to access a document in the address, wherein the document includes at least a portion of the patient information as determined by the condition for access.

17. The computer-implemented method of claim 16, wherein the condition for access is a payment term based on a type of patient information requested and on an identity of a third party making the request, further comprising providing, to the server in the health information exchange, a credential indicative of the identity of the third party.

18. The computer-implemented method of any one of claims 16 and 17, further comprising decrypting the portion of the patient information using the key and displaying the portion of the patient information in a third party user device.

19. The computer-implemented method of any one of claims 16 through 18, wherein the key comprises a private key and a random secret key, further comprising finding a hashing function to decrypt the portion of the patient information based on the private key and the random secret key.

20. The computer-implemented method of any one of claims 16 through 19, wherein the patient information comprises multiple patients, further comprising receiving, from the server in the health information exchange, a list of the patients satisfying a condition in the request, and selecting one or more of the patients from the list, based on the condition.
